(19) United States
(12) Patent Application Publication
(10) Pub. No.: US 2025/0352558 A1
(43) Pub. Date: Nov. 20, 2025

(54) WATER-SOLUBLE CANNABIDIOL COMPOSITIONS AND METHOD FOR PREPARATION OF WATER-SOLUBLE CANNABIDIOL

(71) Applicant: Teerapol SRICHANA, Hat Yai, Songkhla (TH)

(72) Inventor: Teerapol SRICHANA, Hat Yai, Songkhla (TH)

(21) Appl. No.: 18/876,277

(22) PCT Filed: Aug. 16, 2023
(86) PCT No.: PCT/TH2023/000019

§ 371 Date: Dec. 18, 2024

(30) Foreign Application Priority Data
Nov. 30, 2022 (SG) 10202260255W

(51) Int. Cl. A61K 31/00; A61K 9/00; A61K 9/107; A61K 9/16; A61P 29/00; A61P 31/14

(52) U.S. Cl. CPC A61K 31/658; A61K 9/0043; A61K 9/1075; A61K 9/1641; A61K 9/1652; A61K 9/1694; A61P 29/00; A61P 31/14

(57) ABSTRACT

The present invention relates generally to a method of formulation of cannabidiol (CBD) a potential anti-inflammatory drug derived from lemon peel (d-limonene). More, particularly, the present invention relates to the enhancement of lipid soluble CBD into water soluble form in order to improve the formulation of the active component while apply in human for medication. The present invention further provides the therapeutic application of the new formulation which either neutralize and/or decreases the capability of the production of cytokines by activated immune cells during inflammatory process and hence alleviate the sufferings of illness like COVID-19 caused by SARSCOV2.

### FIELD OF THE INVENTION

This disclosure relates to cannabidiol compositions. In particular, this disclosure relates to water-soluble cannabidiol (CBD) compositions, including methods for preparing water-soluble CBD compositions and its application.

### BACKGROUND OF THE INTERVENTION

Cannabidiol (CBD) is a cannabinoid predominantly found in the flowering plants of the genus cannabis, including Cannabis sativa, Cannabis indica, and Cannabis ruderalis. CBD is an active compound acting on cannabinoid receptors, CB1 and CB2. It produces pharmacological effects, mainly in the central nervous system and the immune system and does not cause intoxication or euphoria (the “high”) that comes from tetrahydrocannabinol (THC). The Clinical research on CBD included studies related to anxiety, cognition, movement disorders, pain, and immunity. CBD can be taken by inhaling cannabis smoke or vapor, by mouth, and as an aerosol spray.

In the United States, the cannabidiol drug Epidiolex was approved by the Food and Drug Administration in 2018 for the treatment of two epilepsy disorders and the 2018 United States Farm Bill removed hemp and hemp extracts (including CBD) from the Controlled Substances Act, however, the marketing and sale of CBD formulations for medical use while as an ingredient in dietary supplements or manufactured foods remains illegal under FDA regulation, as of 2021.

One of the major challenges in formulating CBD products is that CBD is poorly soluble in aqueous solutions and to be effective of CBD in human body, it must be present in the form of a solution at the site of its absorption. The major disadvantage of the currently available CBD products is of its lipid-based formulation, which puts an obstacle not only to the formulation but also in bioavailability of the said component once is taken by patients. Formulation of CBD as solutions, eye drops, injections, aerosols, and so on, needs to be in an aqueous solution of the highly lipophilic CBD.

CBD is detected by the receptor immune cells such as monocytes, macrophage, neutrophils etc is know as CB2. The role of this receptor in modulating inflammation and immune response is well documented in fact in several settings. The stimulation of CB2 receptors is known to limit the release of pro-inflammatory cytokines, shift the macrophage phenotype towards the anti-inflammatory M2 type and enhance the immune-modulating properties of mesenchymal stromal cells. We hypothesized that CB2 receptor is more readily utilized by our invented formulation better than other form and can be a therapeutic target in COVID-19 pandemic emergency.

The immune system has complex mechanisms to fulfill its function and respond to a variety of signaling molecules including hormones, neurotransmitters, and specific lipids, such as endocannabinoids (eCBs). The biological effects of cannabinoids are mediated through the activation of G-protein-coupled cannabinoid (CB) receptors. The endocannabinoid system (ECS) includes the CB1 and CB2 the endogenous cannabinoids, and the enzymes for their metabolism. CB1 is mostly expressed in the central nervous system and is strongly associated with the psychoactive effects of cannabinoids. CB1 is also expressed at low levels in peripheral tissues. Instead, CB2 is highly expressed by immune cells (B cells, natural killer cells, monocytes, neutrophils, CD8 lymphocytes, CD4 lymphocytes) and in several organs and tissues such as liver, spleen, nasal epithelium, thymus, brain, lung and kidney.

There is thus a current demand of a new formulation of CBD. In particular, there is a need of methods of preparing water-soluble CBD compositions. The current invention is mostly related with the new and robust method of formulation which makes CBD more soluble and absorbable once adminstrated in human body.

### BRIEF DESCRIPTION OF DRAWINGS

FIG. 1 The Source of CBD and is extracted from the peel of Lemon.

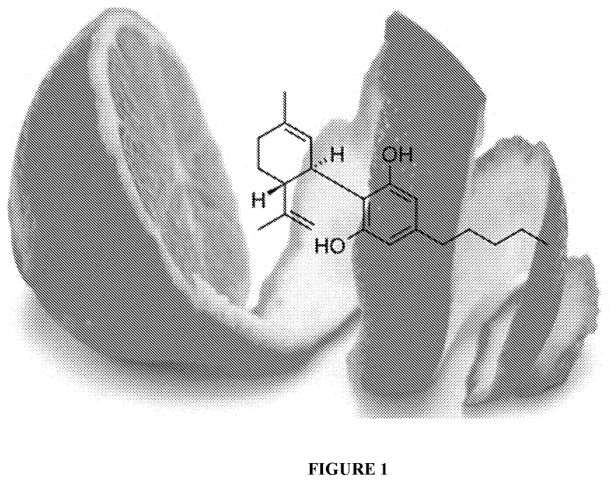

FIG. 2 This image is a model of the CB2 receptor based on experimental data. The red portion is a binding agent used in experimentation to find out where the binding sites on the receptor are. Image courtesy of the Journal of Chemical Information and Modeling.

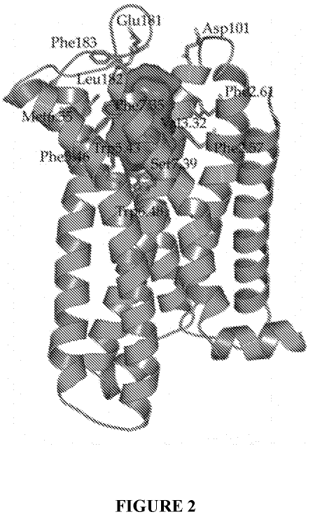

FIG. 3 Schematic diagram of presenting the possible role of immune cells in releasing cytokines during COVID-19 inflammatory process.

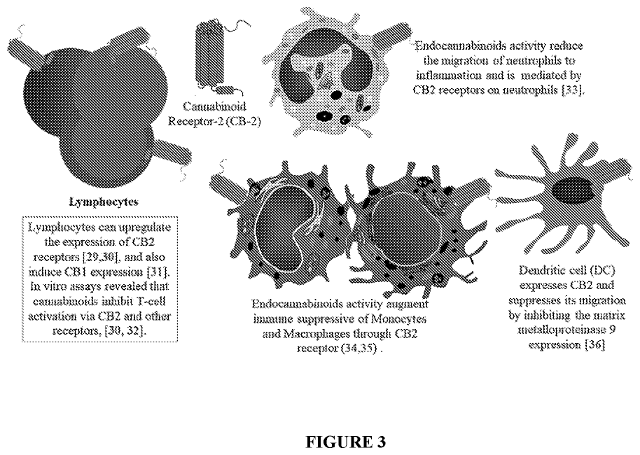

FIG. 4 Differential scanning calorimetry thermograms of the water-soluble composition comprised of CBD, beta-cyclodextrin, and poloxamer 407.

FIG. 5 Differential scanning calorimetry thermograms of the physical mixture comprised of CBD, beta-cyclodextrin, and poloxamer 407.

FIG. 6 Size distribution by the number of the CBD water-soluble composition dispersed in water determined using the dynamic light scattering technique. Data are represented as mean±SD (n=6).

FIG. 7 Apparent zeta potential of the CBD water-soluble composition dispersed in water determined using the dynamic light scattering technique. Data are represented as mean±SD (n=6).

FIG. 8 Percentage of cell viability of the water-soluble CBD composition on human nasal septum tumor epithelial cells (RPMI 2650). Data are expressed as mean±SD (n=4).

FIG. 9 Percentage of cell viability of the water-soluble CBD composition on mouse macrophage-like cells (RAW264.7). Data are expressed as mean±SD (n=4).

FIG. 10 The amounts of tumor necrosis factor-alpha (TNF-α) produced from mouse macrophage-like cells (RAW264.7) in the untreated control group; after being challenged with the CBD aqueous nasal spray (sample group); after being challenged with the sample before being stimulated with S-RBD (prevention group), after being challenged with the sample after being stimulated with S-RBD (treatment group); and after being stimulated with the S-RBD of the SARS-COV-2 virus. Data are expressed as mean±SD (n=3).

FIG. 11 The amounts of interleukin-1 beta (IL-1B) produced from mouse macrophage-like cells (RAW264.7) in the untreated control group; after being challenged with the CBD aqueous nasal spray (sample group); after being challenged with the sample before being stimulated with S-RBD (prevention group), after being challenged with the sample after being stimulated with S-RBD (treatment group); and after being stimulated with the S-RBD of the SARS-COV-2 virus. Data are expressed as mean±SD (n=3).

FIG. 12 The amounts of interleukin-6 (IL-6) produced from mouse macrophage-like cells (RAW264.7) in the untreated control group; after being challenged with the CBD aqueous nasal spray (sample group); after being challenged with the sample before being stimulated with S-RBD (prevention group), after being challenged with the sample after being stimulated with S-RBD (treatment group); and after being stimulated with the S-RBD of the SARS-COV-2 virus. Data are expressed as mean±SD (n=3).

FIG. 13 Schematic diagram of presenting the possible role of immune cells in releasing cytokines during COVID-19 inflammatory process.

FIG. 14 The schematic diagram of application of CBD in COVID-19 infected patients and mechanism of its action against cytokines storm.

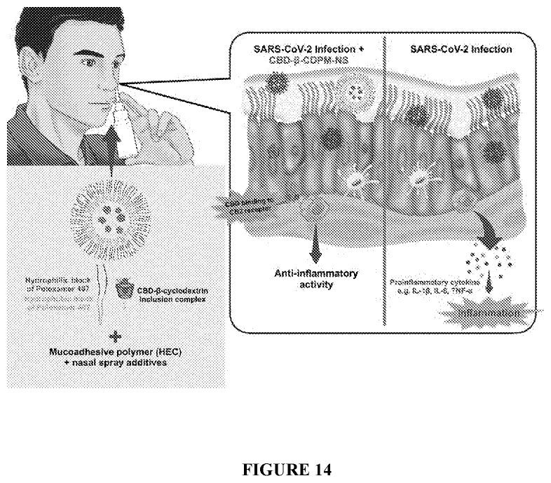

### DETAILED DESCRIPTION OF THE INVENTION

The invention disclosed herein is water-soluble compositions of CBD, cyclodextrin, and poloxamers. In one embodiment, the compositions disclosed herein are used for preparing other formulations, particularly nasal spray formulations. In some aspects, the invention disclosed herein also includes methods for the preparation of water-soluble CBD compositions. In one particularly promising aspect, the compositions and formulations disclosed herein are used to prevent and/or alleviate the inflammatory responses caused by COVID-19.

As used herein, the term “cannabidiol” refers to a cannabinoid predominantly found in the plants of the genus cannabis. In one embodiment, CBD can be a classical, non-classical, or synthetic analog at various levels of purity. For example, CBD can be at least 80% pure, 80-85% pure, 85-90% pure, 90-95% pure, 95-99% pure, 99-99.9% pure, or greater than 99.9% pure. The structural formula of CBD is given below.

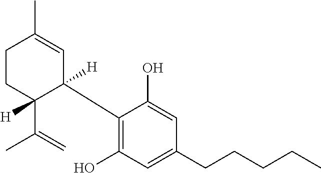 [SMILES: C=C(C)[C@@H]1CCC(C)=C[C@H]1c1c(O)cc(CCCCC)cc1O]

The CBD used in this invention is extracted from Lemon Peel (Fig-1) and obtained from AVS innovation Bangkok, Thailand. Hence zero chance of cotamination of THC (Tetrahydracanabidiol).

In current invention the CBD is complexed with a water-soluble cyclodextrin and forms micelles with a poloxamer to create the water-soluble CBD compositions. As used herein, the term “water hydro-soluble” means that at least 1.0 g of the substance can be dissolved in 100 ml of water at 20° C.

Cyclodextrins are ring-shaped oligosaccharides, consisting of glucose subunits. In this preferred embodiment, the cyclodextrins used herein are beta-cyclodextrin and hydroxypropyl-beta-cyclodextrin. They are preferred because the relative size of their interiors and CBD are quite similar and they are safe for human ingestion and application. As used herein, the term “beta-cyclodextrin” refers to cyclodextrins that contain 7 glucose subunits in a ring including hydroxypropyl-beta-cyclodextrin. The structural formula of beta-cyclodextrin is given below.

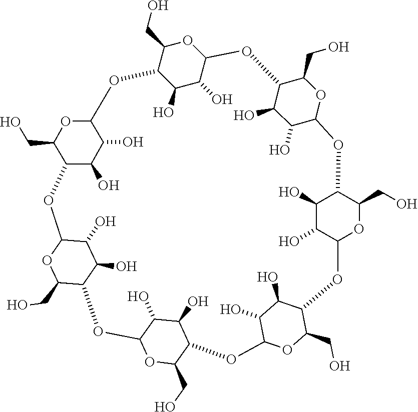 [SMILES: OC[C@H]1OC2O[C@H]3[C@H](O)[C@@H](O)C(O[C@H]4[C@H](O)[C@@H](O)C(O[C@H]5[C@H](O)[C@@H](O)C(O[C@H]6[C@H](O)[C@@H](O)C(O[C@H]7[C@H](O)[C@@H](O)C(O[C@H]8[C@H](O)[C@@H](O)C(O[C@H]1[C@H](O)[C@H]2O)O[C@@H]8CO)O[C@@H]7CO)O[C@@H]6CO)O[C@@H]5CO)O[C@@H]4CO)O[C@@H]3CO]

As used herein, the term “poloxamer” refers to nonionic triblock copolymers formed by polar (polyethylene oxide) and non-polar (polypropylene oxide) blocks. In one embodiment, the poloxamers used herein are water-soluble which include but are not limited to poloxamer 407 (Pluronic® F-127), poloxamer 338 (Pluronic® F-108), poloxamer 188 (Pluronic® F-68), poloxamer 237 (Pluronic® F-87), or any combination thereof. Poloxamers in aqueous solutions, poloxamers, the amphiphilic nature of the copolymers leads their molecules to aggregate and form micelles with a hydrophobic interior constituted by polypropylene oxide blocks (b) and a hydrophilic exterior constituted by polyethylene oxide blocks (a). As used herein, the term 10 “micelle” refers to a loosely bound aggregation of amphiphilic molecules dispersed in a solution, found in a spherical form, and often having a hydrophobic pocket. The poloxamer micelles are nano-sized structures, usually between 10-200 nm that appear at the critical micellization concentration and at critical micellization temperature. The general formula of poloxamers is given below.

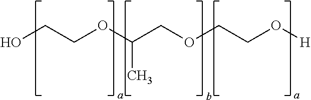

Within the context of this disclosure, CBD is formulated with beta-cyclodextrin and poloxamer to increase the aqueous solubility and bioavailability of poorly soluble CBD.

In one embodiment, at a molecular level, the water-soluble CBD compositions disclosed herein comprise of approximately 1:1:0.03, approximately 1:1:0.06, approximately 1:1:0.09, approximately 1:1:0.12, approximately 1:1:0.15, approximately 1:1:0.18, or approximately 1:1:0.21 molar ratio between the CBD, beta-cyclodextrin, and poloxamer. The method for the preparation of the above composition comprises the steps of
a) Dissolving CBD in ethanol, resulting in an alcoholic CBD solution,
b) Dissolving beta-cyclodextrin in water at 70-90° C. and stirring vigorously for approximately 30-60 min, resulting in an aqueous beta-cyclodextrin solution,
c) Dissolving poloxamer in water at 70-90° C. and stirring for approximately 30-60 min, resulting in an aqueous poloxamer solution,
d) Mixing the aqueous solution of step b) with the aqueous solution of step c), resulting in an aqueous beta-cyclodextrin and poloxamer solution,
e) Mixing the alcoholic CBD solution of step, a) with the aqueous beta-cyclodextrin and poloxamer solution of step d) at 70-90° C. (at the water to alcohol ratio preferably of 90: 10% v/v) and stirring continuously for approximately 30-60 min, resulting in CBD-cyclodextrin-poloxamer mixture,
f) Subjecting the mixture in step d) to freeze-drying at −40 to 20° C., resulting in a water-soluble CBD composition.

An example of the water-soluble CBD composition described herein is an off-white amorphous powder at room temperature. In one preferred embodiment, the water-soluble CBD compositions described herein may be formulated for intranasal delivery with a nasal spray. For example, the aqueous nasal spray formulations in Example 2.

The present invention is directed towards a therapeutic application of one or more pathological conditions in particular the condition where body immune cells release chemicals called cytokines to an amount sufficient for the damaging of health cells in affected organ/organs. The therapeutic component is constructed in a manner to allow for the controlled release and its interaction with toxic chemicals such as cytokines. Hence, the present invention has application in medical treatment such SARS COV2.

As will be understood by the ordinary skilled artisan upon reading the specification, the analyte can be any specific substance or component that one is desirous of treating, inhibiting and/or neutralizing chemicals such as cytokines during pathological conditions like SARS COV2 infection (COVID-19).

Coronavirus disease (COVID-19) is an infectious disease caused by the SARS-COV-2 virus which the World Health Organization (WHO) declared the outbreak a public health emergency of international concern on 30 Jan. 2020 and a Pandemic on 11 Mar. 2020. As of 9 Nov. 2022, the pandemic had caused more than 638 million cases and 6.6 million confirmed deaths, making it one of the deadliest in history. The United States, India, and Brazil have been among the countries hardest-hit by the pandemic.

Most people who fall sick with COVID-19 will experience mild to moderate symptoms and recover without special treatment. However, some will become seriously ill and require medical attention. Infected persons are typically contagious for 10 days, and can spread the virus even if they do not develop symptoms. Mutations have produced many variants with varying degrees of infectivity and virulence.

Symptoms of COVID-19 are variable, ranging from mild symptoms to severe illness. Common symptoms include headache, loss of smell and taste nose congestion and runny nose cough, muscle pain, sore throat fever, diarrhea, and breathing difficulties. People with the same infection may have different symptoms, and their symptoms may change over time. Three common clusters of symptoms have been identified: one respiratory symptom cluster with cough, sputum, shortness of breath, and fever; a musculoskeletal symptom cluster with muscle and joint pain, headache, and fatigue; a cluster of digestive symptoms with abdominal pain, vomiting, and diarrhea. In people without prior ear, nose, and throat disorders, loss of taste combined with loss of smell is associated with COVID-19 and is reported in as many as 88% of cases.

COVID-19 can trigger a cytokine storm in pulmonary tissues through hyperactivation of the immune system and the uncontrolled release of cytokines. The phrase “cytokine storm” is a descriptive term to encompass a variety of events that may ultimately result in multi-organ failure and death. Cytokine storms can cause a severe clinical complication known as acute respiratory distress syndrome (ARDS). ARDS is induced by an excessive immune response rather than the viral load. Growing evidence is being documented on the possible role of the pro-inflammatory cytokines released by immune cells (Fig-11) in COVID-19 pathogenesis and related complications.

With such a reason, management of the cytokine release syndrome (CRS) and preventing subsequent infections may be an intriguing approach for COVID-19 therapy. Accordingly, there is an imperative need to develop anti-inflammatory agents for COVID-19 patients who develop CRS. Identifying the underlying mechanisms can aid in developing therapeutic strategies and speed up recovery. Several clinical trials are ongoing to investigate novel supportive care and interventions to cure this infection. To date, no definite and effective therapeutic agents are available. However, some supportive therapeutic interventions and anti-viral agents with limited efficacy are in hand to decrease the sufferings of COVID-19 infected patients and mitigate its symptoms. In this regard, anti-inflammatory therapy in patients with COVID-19 may be a promising intervention for COVID-19-related pneumonia. Our present invented therapeutic agent CBD will address the current understanding of cytokines-induced alterations in COVID-19, focusing on interleukin (IL)-6, IL-1, IL-17, and tumor necrosis factor (TNF) and further enhances the scope of therapeutic strategies to rescue affected patients with severe COVID-19.

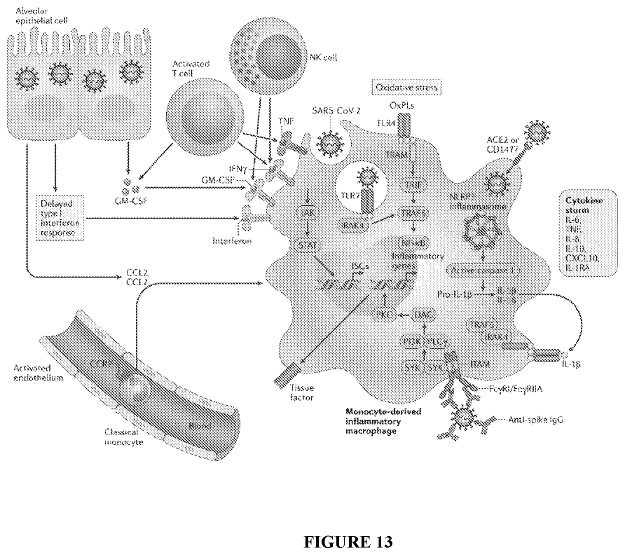

The stimulation of CB2 could control the inflammatory cascade in several checkpoints, considering its capability to reduce the production of a large number of cytokines, contrarily to the extremely selective action of monoclonal antibodies directed against a specific interleukin. On the other hand, CB2 receptor (Fig-12) stimulation has a well-documented immunosuppressive effect by reducing immune cells proliferation and production of antibodies; thus, it could be greatly beneficial in containing the exacerbated inflammatory response in COVID-19 patients (FIG. 13)

The therapeutic applications of the water-soluble CBD compositions described herein may include every informed ailment that our invented formulation of CBD can be used to treat COVID-19 patients as it can have better scope of utilization of CB2 receptor in nasal cavity where SARSCOV2 anchor first and initiate multiplication cycle. One particularly promising aspect seems to be the prevention and/or alleviation of the inflammatory responses caused by SARSCOV2 infection in humans.

### Examples

The following Examples are provided for the purpose of illustration only.

### Example 1: Components and Composition per 1000 grams of CBD as shown in Table-1

A water-soluble CBD composition (approximately 1:1:0.03 molar ratio between CBD, beta-cyclodextrin, and poloxamer 407) is prepared using the following ingredients to make a total of 1000 g of the mixture.

TABLE 1
The composition of water-soluble CBD

| Ingredients | Grams (g) |
|---|---|
| CBD | 7.9 |
| Beta-cyclodextrin | 28.4 |
| Poloxamer 407 | 9.9 |
| Ethanol | 78.0 |
| Water | 875.8 |

The composition is prepared as follows:
a) Dissolve 7.9 g of the CBD in 78.0 gm of the ethanol and stirred until a clear solution is obtained,
b) In a separated vessel, disperse 28.4 g of the beta-cyclodextrin in about 400.0 g of the water at 70° C., by means of slow addition while stirring at a high-shear rate, and stir for at least 60 min or until a clear solution is obtained,
c) In a separated vessel, disperse 9.9 g of the poloxamer 407 in the remaining water at 70° C., by means of slow addition while stirring at a low-shear rate, and stir for at least 60 min or until a clear solution is obtained,
d) Mix the aqueous solution of step b) with the aqueous solution of step c),
e) Add the ethanol solution of step a) to the aqueous solution of step d) at 70° C., and stir at a low-shear rate for at least 30 min or until a uniform dispersion is formed, resulting in a total of 1000 g of the mixture,
f) Subject the mixture in step e) to freeze-drying from pre-freezing at −40° C. for 24 h followed by primary drying at −10° C., 0.633 mbar for 24 h, and, secondary drying at 10° C. for 4 h and then 20° C. for 20 h, resulting in a water-soluble CBD composition.

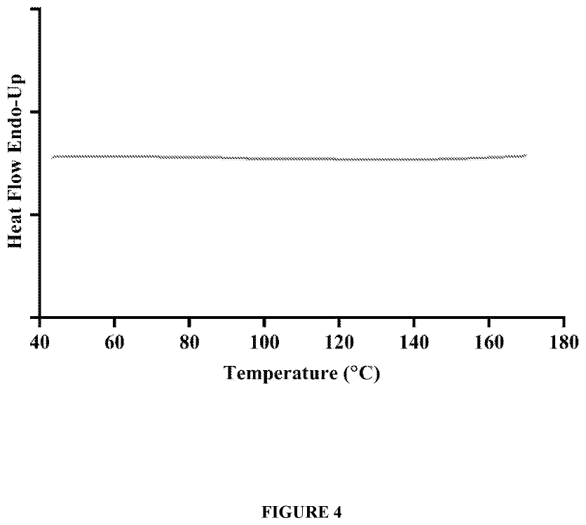

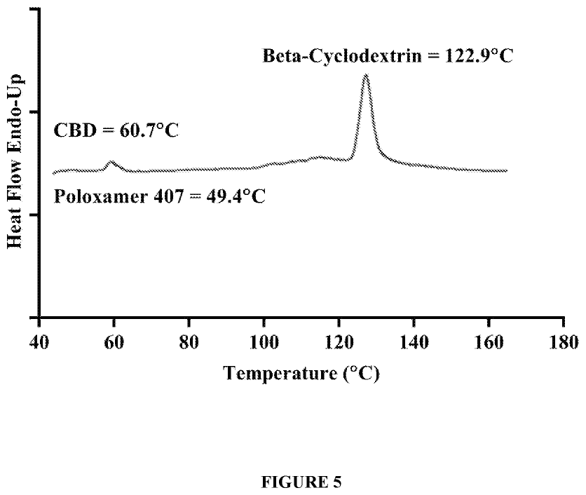

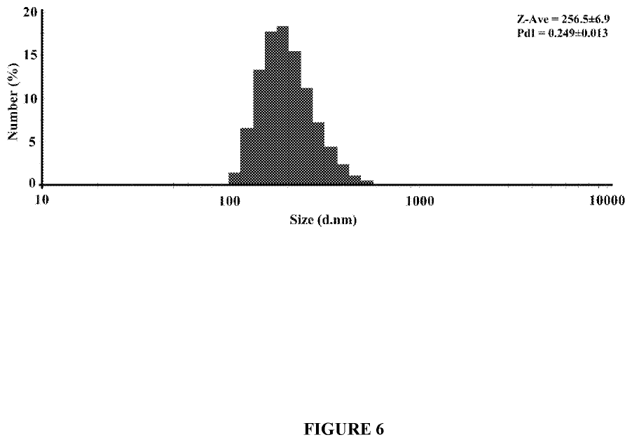

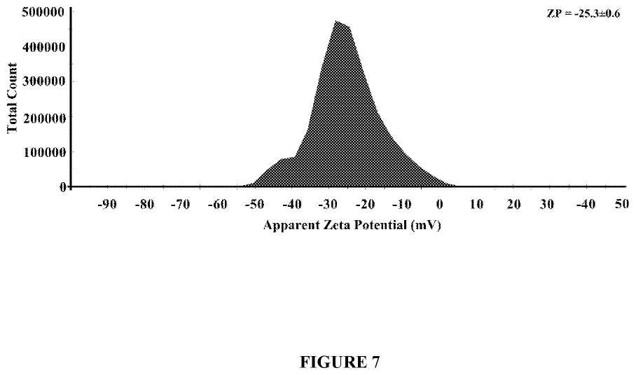

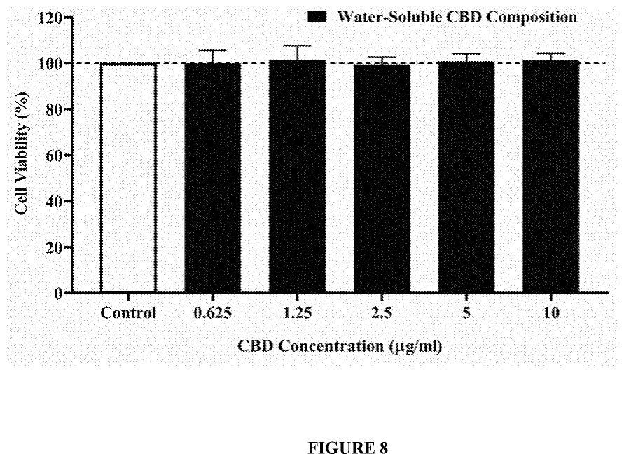

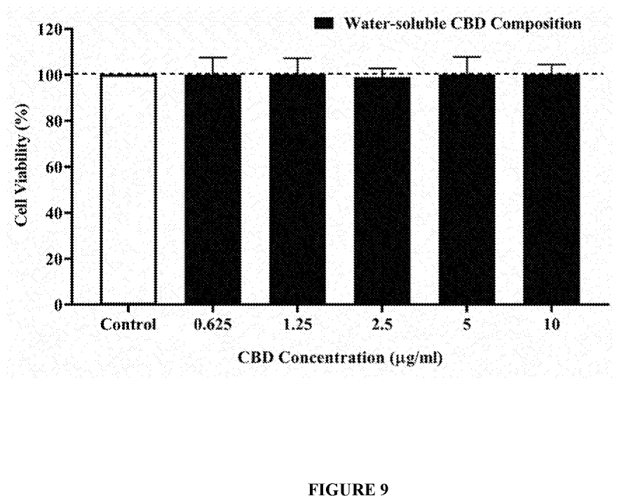

An example of the water-soluble CBD composition described herein is an off-white amorphous powder at room temperature, with 4.79% w/w of moisture content. The yield of the water-soluble CBD composition was 90.9% (42.0 g) of the total weight of the solid ingredients (46.2 g) in the composition. The differential scanning calorimetry thermogram in FIG. 4 shows that the water-soluble CBD composition is an amorphous solid while the differential scanning calorimetry thermogram in FIG. 5 shows the melting characteristic peaks of poloxamer 407, CBD, and beta-cyclodextrin in a physical mixture at 49.4° C., 60.7° C., and 122.9° C., respectively. The solubility of the water-soluble CBD composition was approximately 1.0 g/100 ml. at 20° C. As shown in FIG. 6, the size of the water-soluble CBD composition was 256.5±6.9 nm and its polydispersity index was 0.249±0.019 (n=6). The apparent zeta potential of the water-soluble CBD composition was −25.3±0.6 mV (n=6) when dispersed in water as shown in FIG. 7. At a concentration of 0.625 μg/ml, 1.25 μg/ml, 2.5 μg/ml, 5 μg/ml, and 10 μg/ml, the water-soluble CBD composition shows 100% cell viability for RPMI 2650 (FIG. 8) and RAW264.7 (FIG. 9) cell lines.

### Example 2: Formulations of aqueous nasal spray of CBD

The aqueous nasal spray formulations comprised of the water-soluble CBD composition are prepared using the following ingredients where concentrations are weight percentages and shown in Table 2.

TABLE 2
The composition and formulation of nasal spray of CBD

| Purpose | Ingredient | % w/w |
|---|---|---|
| Active ingredient | Water-soluble CBD | 0.30-1.00 |
| | composition | |
| Stabilizing agent | Microcrystalline cellulose | 1.35-2.70 |
| Rheology-modifying agent | Sodium carboxymethyl | 0.15-0.30 |
| | cellulose | |
| Rheology-modifying agent | Polyvinylpyrrolidone K30 | 3.00-5.00 |
| Rheology-modifying agent | Polyethylene glycol 1500 | 3.00-5.00 |
| Humectant | Glycerin | 0.55-1.30 |
| Buffering agent | Sodium phosphate, dibasic | 0.90-1.00 |
| Buffering agent | Sodium phosphate, monobasic | 0.45-0.55 |
| Chelating agent | Disodium EDTA | 0.03-0.05 |
| Preservative | Benzyl alcohol | 0.25-1.00 |
| Preservative | Benzalkonium chloride | 0.02-0.10 |
| Solvent | Water | 82.00-90.00 |

An example of the aqueous nasal spray formulation comprises of the water-soluble CBD composition was prepared using the above ingredients and concentrations.

### Example-3. Preparation of droplets from aqueous nasal spray composition

The droplet size distribution of the CBD aqueous nasal spray is shown in Table-3.

TABLE 3
The size of droplets and their distances
that cover during application.

Nozzle to laser distance (mm)

| Parameter | 30 | 60 |
|---|---|---|
| Dv(10) (μm) | 34.0 ± 2.6 | 33.3 ± 1.3 |
| Dv(50) (μm) | 102.5 ± 4.6 | 91.7 ± 5.0 |
| Dv(90) (μm) | 221.7 ± 10.6 | 201.1 ± 9.8 |
| Span | 1.832 ± 0.103 | 1.832 ± 0.072 |

Data are represented as mean ± SD (n = 6)

The CBD aqueous nasal spray was evaluated for its ability to inhibit the excessive pro-inflammatory cytokine production from or reduce the inflammatory cytokine activity of the RAW264.7 (macrophage-like) cell line in response to the inflammatory stimulant-a recombinant spike receptor-binding domain (S-RBD) of the SARS-COV-2 virus. S-RBD is the viral protein that binds the virus to the ACE-2 receptor on the human macrophages, resulting in the excessive production of pro-inflammatory cytokines, inflammation, and tissue injury. S-RBD was used as an antigen to stimulate the macrophages to produce the pro-inflammatory cytokines, including tumor necrosis factor-alpha (TNF-α), interleukin-1 beta (IL-1B), and interleukin-6 (IL-6).

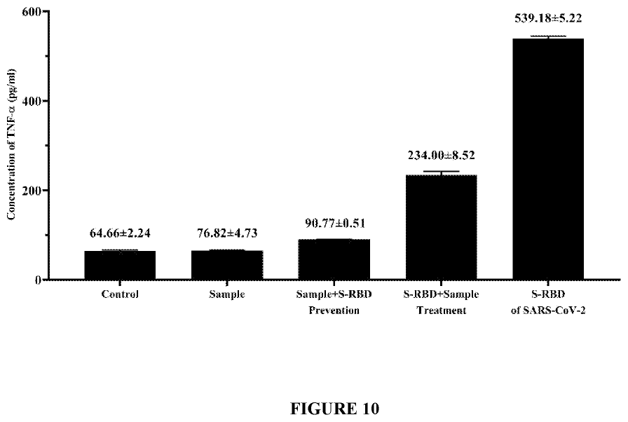

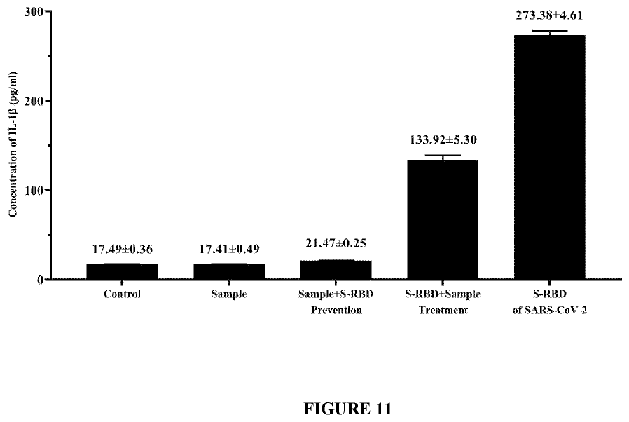

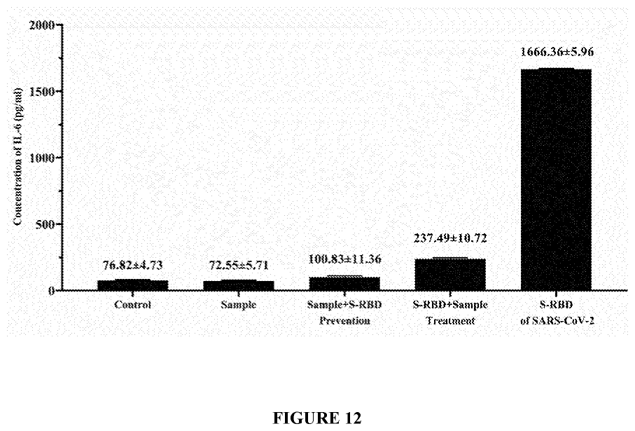

The S-RBD of the SARS-COV-2 virus induced RAW264.7 cells to produce very high amounts of TNF-α, IL-1B, and IL-6, which are 539.18+5.22 pg/ml (FIG. 10), 273.38+4.61 pg/ml (FIG. 11), and 1666.36-5.96 pg/ml (FIG. 12), respectively (n=3). As shown in FIG. 10, FIG. 11, and FIG. 12, the CBD aqueous nasal spray (sample group) did not stimulate RAW264.7 cells to produce any pro-inflammatory cytokines as the detected amounts of cytokine were very low and similar to the amounts of cytokine in the untreated control group. There is a significant reduction in amounts of cytokine when the S-RBD stimulated RAW264.7 cells were treated with the sample (treatment group). The amounts of TNF-α, IL-1β, and IL-6 were 234.00±8.52 pg/ml (FIG. 10), 133.92±5.30 pg/ml (FIG. 11), and 237.49+10.72 pg/ml (FIG. 12), respectively (n=3). However, the greatest reduction in amounts of cytokine was obtained when the sample was challenged to RAW264.7 cells before being stimulated with S-RBD (prevention group). The amounts of TNF-α, IL-1β, and IL-6 were 90.77+0.51 pg/ml (FIG. 10), 21.47+0.25 pg/ml (FIG. 11), and 100.83+11.36 pg/ml (FIG. 12), respectively (n=3).

The reduced amounts of cytokines obtained from both treatment and preventive groups were significantly lower than the amounts of cytokine produced in the S-RBD stimulated group. This suggested that the CBD aqueous nasal spray can be used for both prevention and alleviation of the inflammatory responses caused by SARS-COV-2 infection in humans.

### BEST MODE FOR CARRYING OUT THE INVENTION

As already mentioned in the full disclosure of invention.

1. A water-soluble composition comprising a CBD, beta-cyclodextrin, and a poloxamer.

2. The composition according to claim 1, wherein the CBD comprises a classical analog, a non-classical analog, or a synthetic analog of CBD.

3. The composition according to claim 1, wherein the levels of purity of the CBD are at least 80% pure, 80-85% pure, 85-90% pure, 90-95% pure, 95-99% pure, 99-99.9% pure, or greater than 99.9% pure.

4. The composition according to claim 1, wherein the beta-cyclodextrin comprises a beta-cyclodextrin or hydroxypropyl-beta-cyclodextrin.

5. The composition as claimed in claim 1, wherein the poloxamer comprises poloxamer 407, poloxamer 338, poloxamer 188, poloxamer 237, or any combination thereof.

6. The composition as claimed in claim 1, wherein the composition comprises micelles.

7. The composition as claimed in claim 1, wherein the composition is an amorphous powder.

8. The composition as claimed in claim 1, wherein the solubility of the composition is greater than 1.0 g/100 ml in water at 20° C.

9. The composition as claimed in claim 1, wherein the particle size of the composition is in a range of 10-300 nm when dispersed in water.

10. The composition as claimed in claim 1, wherein the composition is not cytotoxic to the human nasal epithelial cells and macrophages.

11. The composition as claimed in claim 1, wherein the composition does not stimulate the macrophages to produce pro-inflammatory cytokines including but not limited to TNF-α, IL-1β, and IL-6.

12. The composition as claimed in claim 1, wherein the composition has a molar ratio between the CBD, the beta-cyclodextrin, and the poloxamer of approximately 1:1:0.03, approximately 1:1:0.06, approximately 1:1:0.09, approximately 1: 1:0.12, approximately 1:1:0.15, approximately 1:1:0.18, or approximately 1:1:0.21.

13. The composition according to claim 1, wherein the composition is formulated as a nasal spray.

14. A method of producing a water-soluble composition comprising a CBD, a beta-cyclodextrin, and a poloxamer, comprises the steps of mixing a CBD, a beta-cyclodextrin, and a poloxamer to form a liquid mixture, and freeze-drying the liquid mixture to form a powder.

15. The method according to claim 14, wherein the CBD, beta-cyclodextrin, and poloxamer are mixed with both in water and in alcohol.

16. The method according to claim 15, wherein the water and alcohol are removed by freeze-drying.

17. The method as claimed in claim 14, wherein the first temperature is from 70° C. to 90° C.; and wherein the second temperature is from −40° C. to 20° C.

18. A method of using a water-soluble composition comprising a CBD, a beta-cyclodextrin, and a poloxamer, to prevent and/or alleviate the inflammatory responses caused by the SARS-COV-2 infection.

19. The method according to claim 18, wherein the inflammatory responses in humans include the release of pro-inflammatory cytokines including but not limited to TNF-α, IL-1β, and IL-6.
